(19) United States
(12) Patent Application Publication
(10) Pub. No.: US 2008/0262017 A1
(43) Pub. Date: Oct. 23, 2008

(54) Sugar-Free Storage-Stable Antihistaminic Syrups

(76) Inventors: Sergio R. Ulloa, Del. Tialpan (MX); Jose de Jesus Mateo Villacampa Ramos, Col. Del Valle. Del. B. Juarez (MX); Luis Javier Juarez Vargas, Delegacion: Gustavo A. Madero (MX)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/769,351

(22) Filed: Jun. 27, 2007

Related U.S. Application Data
(60) Provisional application No. 60/817,312, filed on Jun. 29, 2006.

(51) Int. Cl. A61K 31/4545; A61P 37/08

(52) U.S. Cl. 514/290

(57) ABSTRACT

New and improved storage-stable sugar-free antihistaminic syrups are disclosed.

### REFERENCE TO RELATED APPLICATION

This application claims the benefit of priority to U.S. Provisional Patent Application 60/817,312 filed Jun. 29, 2006, the entire disclosure of the priority application is hereby incorporated by reference.

### BACKGROUND OF THE INVENTION

Citation of or reference to any application or publication in this Section or any Section of this application is not an admission that such document is available as prior art to the present invention.

The present invention pertains to the field of liquid pharmaceutical formulations, and more particularly to syrup formulations containing antihistamines.

Syrup formulations are commonly used for delivery of pharmacological agents, particularly where the agents are to be delivered to pediatric patients. Traditional syrups are concentrated solutions of sugar (generally sucrose) in purified water, such as Syrup, NF prepared with 850 grams sucrose and sufficient water to make 1000 mL according to the procedure given in the official monograph at page no 1990 of NF 19 The National Formulary, United States Pharmacopeial Convention, Inc., Rockville, Md. U.S.A., 2000. However, for purposes of the present invention, the term “syrup” will also encompass those liquid formulations having a sweet taste provided wholly or partly by artificial sweeteners for avoidance of dental and medical problems which may be aggravated by higher caloric sweeteners.

As is well appreciated in the art, syrups frequently are flavored, such as with fruit or mint flavors, usually for purposes of masking an unpleasant taste caused by the presence of a dissolved or suspended pharmacologically active substance. A pleasant taste is particularly important when the formulation is intended for ingestion by children. Typical flavoring agents which are commonly used in sweetened pharmaceuticals, foods, candies, beverages and the like are also useful in the present invention; these materials impart flavors such as grape, cherry, citrus, peach, strawberry, bubble gum, peppermint and many others.

An example of a currently marketed syrup contains 1 mg/mL of the antihistamine loratadine, together with citric acid, artificial flavor, glycerin, propylene glycol, sodium benzoate, sucrose and water; this formulation typically has a pH value between about 2 and 4. However, under certain storage conditions involving contact with the air, losses of loratadine content, and a concomitant generation of impurities, have occurred. Similar problems can occur with formulations containing other, chemically related, antihistamines, such as desloratadine.

Likewise, U.S. Pat. No. 6,514,520 discloses an antihistaminic syrup formulation comprising desloratadine and about 0.05 to about 5 mg/mL of an aminopolycarboxylic acid or a salt thereof. However, when stored under dark conditions, a strong pink color has been observed to develop over time. Thus, a dye is used in the marketed formulation.

Consequently, there still exists a need for new syrup formulations for the delivery of antihistamines, such as loratadine or desloratadine, that are storage-stable.

### SUMMARY OF THE INVENTION

Accordingly, it is desired to provide a novel storage-stable syrup formulation of loratadine, desloratadine, or a pharmaceutically acceptable salt thereof, or a combination of two or more thereof, which contains only components recognized as being safe for human ingestion, that are sugar free, clear in color and that are storage-stable.

Accordingly, in one embodiment, there is disclosed an antihistaminic syrup formulation in which the ingredients comprise loratadine, desloratadine, or a pharmaceutically acceptable salt thereof, or a combination of two or more thereof, propylene glycol, sorbitol, sodium citrate dihydrate, citric acid anhydrous, povidone, sucralose, optionally sodium benzoate, and optionally an aminopolycarboxylic acid or salt thereof, wherein the antihistaminic syrup formulation has a pH of greater than about 4.5, said antihistaminic syrup formulation being storage-stable.

In addition, in another embodiment, there is disclosed an antihistaminic syrup formulation in which the ingredients comprise loratadine, desloratadine, or a pharmaceutically acceptable salt thereof, or a combination of two or more thereof, propylene glycol, sorbitol, sodium citrate dihydrate, citric acid anhydrous, monoammonium glycyrrhizinate, optionally sodium benzoate, and optionally an aminopolycarboxylic acid or salt thereof, wherein the antihistaminic syrup formulation has a pH greater than about 4.5, said antihistaminic syrup formulation being storage-stable.

In one preferred embodiment, the present invention also provides a novel storage-stable syrup formulation of loratadine, desloratadine, or a pharmaceutically acceptable salt thereof, or a combination of two or more thereof, which contains only components recognized as being safe for human ingestion, that are sugar free, clear in color and that are storage-stable as well as alcohol free and wherein all excipients are present in a concentration in accordance with the WHO recommendation. In one preferred embodiment, the formulation complies with the World Health Organization's recommendation for acceptable daily intake of propylene glycol, that is, 25 mg or less of propylene glycol for every kilogram of body weight. In a preferred embodiment, propylene glycol is present at about 0.01% to about 35%.

In certain embodiments, the antihistamine is desloratadine or a pharmaceutically acceptable salt thereof. In other embodiments, at least one antihistamine is loratadine or a pharmaceutically acceptable salt thereof. In yet other embodiments, one or more other therapeutic agent(s) listed below herein is (are) included in the antihistaminic syrups.

The present invention also provides methods for treating and/or preventing allergic and inflammatory conditions of the skin or airway passages in a human in need thereof which comprises administering an effective amount of the antihistaminic syrup formulations disclosed herein. In one embodiment, an effective amount of the antihistaminic syrup formulation delivers 25 mg or less of propylene glycol for every kilogram of body weight per day.

### DETAILED DESCRIPTION OF THE DRAWINGS

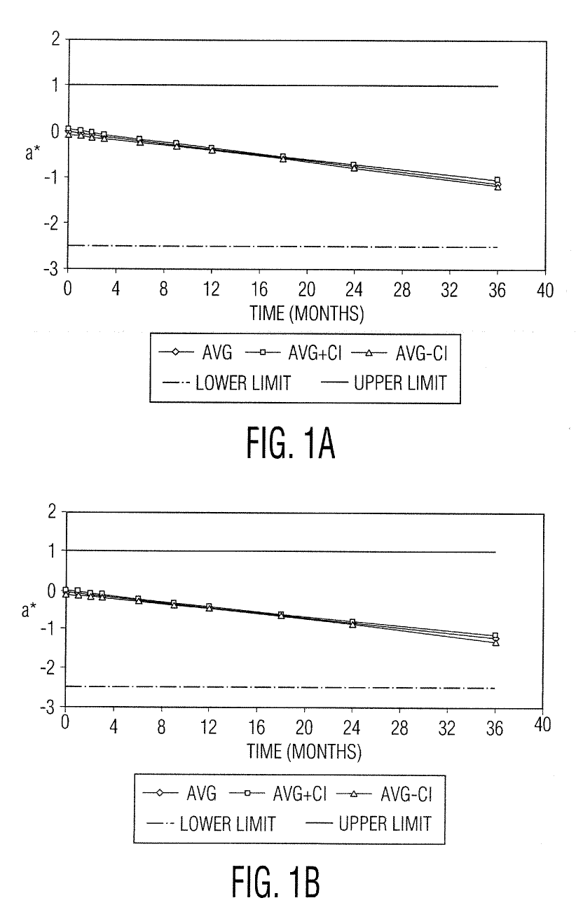

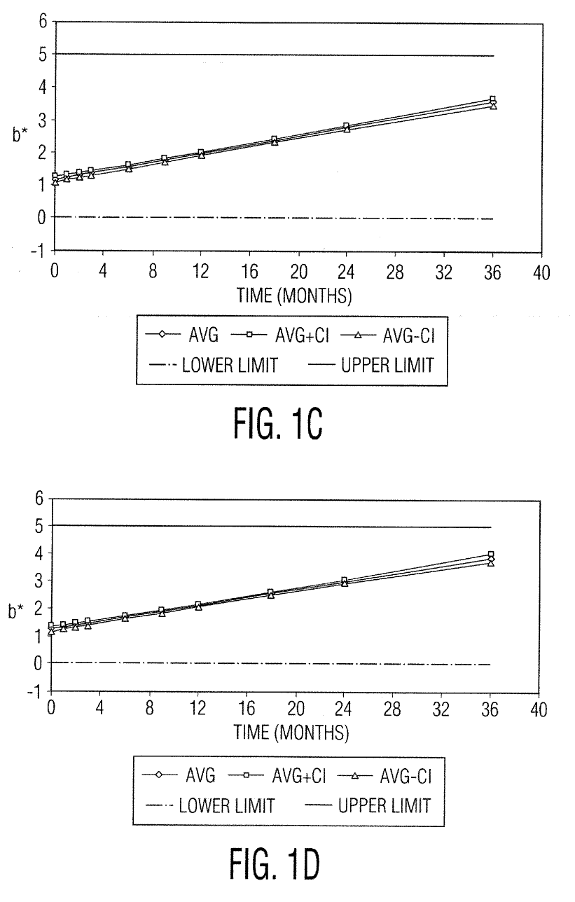

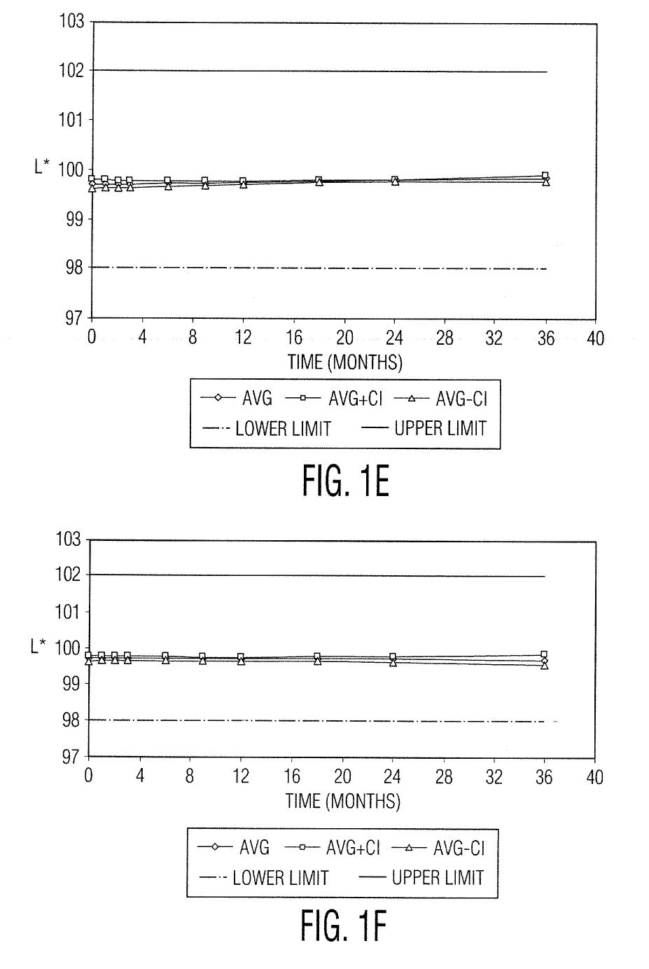

FIG. 1 illustrates the chromaticity coordinates (a, b) and intensity (L*) at 25° C./60% RH or 30° C./65% RH. Specifically, FIGS. 1A and 1B show the chromaticity coordinate (a) at 25° C./60% RH and 30° C./65% RH, respectively; FIGS. 1C and 1D show the chromaticity coordinate (b) at 25° C./60% RH and 30° C./65% RH, respectively, and FIGS. 1E and 1F show the intensity (L*) at 25° C./60% RH and 30° C./65% RH, respectively.

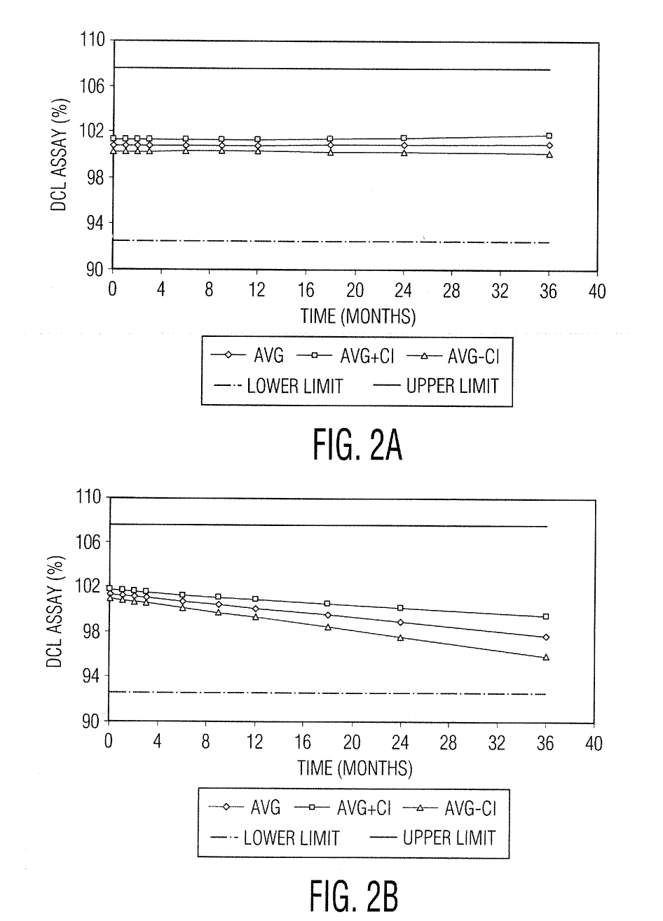

FIG. 2 illustrates the desloratadine stability at 25° C./60% RH or 30° C./65% RH. Specifically, FIGS. 2A and 2B show the desloratadine stability at 25° C./60% RH and 30° C./65% RH, respectively.

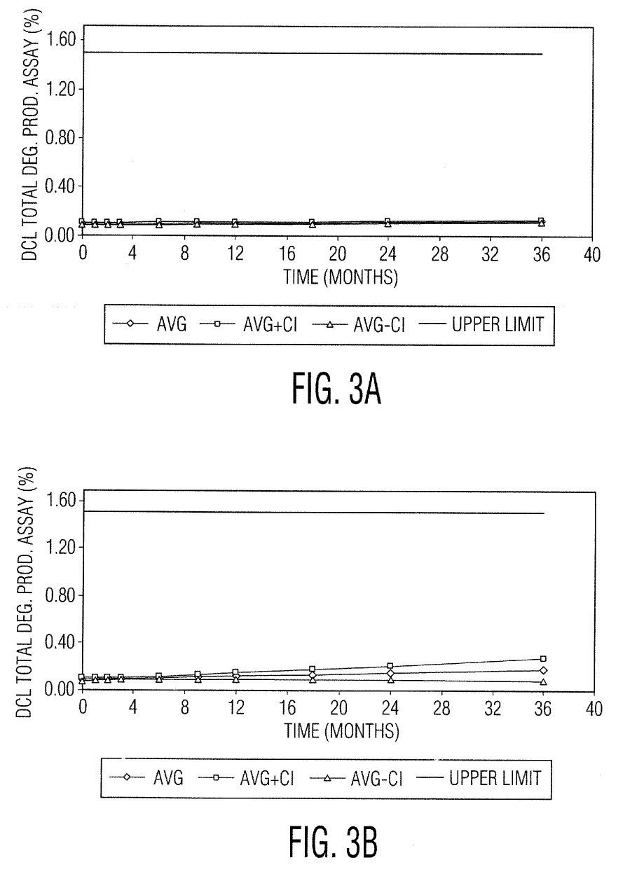

FIG. 3 illustrates the total degradation product stability at 25° C./60% RH or 30° C./65% RH. Specifically, FIGS. 3A and 3B show the total degradation product stability at 25° C./60% RH and 30° C./65% RH, respectively.

### DETAILED DESCRIPTION OF THE INVENTION

The present invention provides an antihistaminic syrup formulation in which the ingredients comprise at least one antihistamine which is loratadine, desloratadine, or a pharmaceutically acceptable salt thereof, or a combination of two or more thereof, propylene glycol, sorbitol, sodium citrate dihydrate, citric acid anhydrous, povidone, sucralose, optionally sodium benzoate, and optionally an aminopolycarboxylic acid or salt thereof, wherein the antihistaminic syrup formulation has a pH of greater than about 4.5, said antihistaminic syrup formulation being storage-stable.

In a preferred embodiment, the pH is between about 4.5 and about 6.5. More preferably, the pH is between about 5 and about 6, more preferably the pH is about 5.5.

In a preferred embodiment, propylene glycol is present at about 0.01 to about 35%.

In one embodiment, the ingredients comprise:

| Ingredient | Concentration (mg/mL) |
|---|---|
| Desloratadine | 0.5 |
| Propylene Glycol | 100 |
| Sorbitol | 150 |
| Sodium Citrate Dihydrate | 2 |
| Citric Acid Anhydrous | 0.64 |
| Povidone | 5 |
| Sucralose | 0.5 |
| Water | q.s. |
| | 1 ml |

In one embodiment, the ingredients further comprise about 0.1 to about 0.5% sodium benzoate.

In one embodiment, the ingredients further comprise about 0.01 to about 5% edetate disodium.

In one embodiment, the antihistaminic syrup formulation has less than 0.2% desloratadine degradation products after storage for 18 months. In one embodiment, the antihistaminic syrup formulation has less than 0.2% desloratadine degradation products after storage for 24 months.

The present invention also provides an antihistaminic syrup formulation in which the ingredients comprise loratadine, desloratadine, or a pharmaceutically acceptable salt thereof, or a combination of two or more thereof, propylene glycol, sorbitol, sodium citrate dihydrate, citric acid anhydrous, mono-ammonium glycyrrhizinate, sodium benzoate, and an aminopolycarboxylic acid or salt thereof, wherein the antihistaminic syrup formulation has a pH greater than about 4.5, said antihistaminic syrup formulation being storage-stable.

In a preferred embodiment, the pH is between about 4.5 and about 6.5. More preferably, the pH is between about 5 and about 6, more preferably the pH is about 5.5.

In a preferred embodiment, propylene glycol is present at about 0.01 to about 35%.

In one embodiment, the ingredients comprise:

| Ingredient | Concentration (mg/mL) |
|---|---|
| Desloratadine, micronized | 0.25 |
| Propylene glycol | 100 |
| Sorbitol | 150 |
| Sodium Citrate Dihydrate | 1.26 |
| Citric Acid Anhydrous | 0.5 |
| Mono-ammonium glycyrrhizinate | 7.5 |
| Water | q.s. |
| | 1 ml |

In one embodiment, the ingredients further comprise about 0.1 to about 0.5% sodium benzoate. In one embodiment, the ingredients further comprise about 0.01 to about 5% edetate disodium.

The present invention also provides methods for treating and/or preventing allergic and inflamatory conditions of the skin or airway passages in a human in need thereof which comprises administering an effective amount of the antihistaminic syrup formulations disclosed herein. In one embodiment, an effective amount of the antihistaminic syrup formulation delivers 25 mg or less of propylene glycol for every kilogram of body weight per day.

Where the term “percent” is used herein, it is intended to represent percent by weight, unless the context clearly evidences otherwise.

Loratadine is disclosed in U.S. Pat. No. 4,282,233 as a nonsedating antihistamine useful, for example, in alleviation of seasonal allergic rhinitis symptoms such as sneezing and itching.

The compound desloratadine is an antihistaminic active metabolite of loratadine. Desloratadine is a white to off-white powder that is slightly soluble in water, but very soluble in ethanol and propylene glycol. It has an empirical formula: C₁₉H₁₉ClN₂ and a molecular weight of 310.8. The chemical name is 8-chloro-6,11-dihydro-11-(4-piperidinylidene)-5H-benzo[5,6]cyclohepta[1,2-b]pyridine. It is available under the Trade names of Clarinex® and Aerius® from Schering Corp., Kenilworth, N.J. U.S. Pat. No. 5,595,997 discloses methods and compositions for treating seasonal allergic rhinitis symptoms using desloratadine.

The antihistaminic syrup formulations of the present invention may also contain one or more other therapeutic agent(s) for obtaining more than one therapeutic result from a single dose. Typical therapeutic agents included with an antihistamine are sympathomimetic amine decongestants, such as pseudoephedrine, phenylpropanolamine or phenylephrine for relief of the upper airway congestion often accompanying disorders such as rhinitis and upper respiratory infections. Antitussives, such as codeine, hydrocodone or dextromethorphan, for relief from coughing, and expectorants such as guaifenesin, for increasing cough productivity, also are included in combination products. H₃ receptor antagonists may also be used in combination with the syrups of the present invention. The histamine H₃ receptor antagonist may be one or more members selected from the group consisting of thioperamide, impromidine, burimamide, clobenpropit, impentamine, mifetidine, clozapine, S-sopromidine, R-sopromidine, ciproxifam, SKF-91486 (3-(imidazole-4-yl)-propylguanidine sulfate), GR-175737 (Clitherow, et al., (1996) Bioorg. Med. 6: 8-833-838), GT-2016 (Tedford, et al., (1995) J. Pharm. Exp. Ther 275(2): 596-604), GT-2331 (Tedford, et al., (1998) Eur. J. Pharmacol. 351(3): 307-11), GT-2394 (Yates, et al., (2000) Soc. Neurosci. Abstr. 26: 279.), JB98064 (Linney, et al., (2000) J. Med. Chem. 43: 2362-2370), UCL-1199 (Ganellin, et al., (1995) J. Med. Chem. 38(17): 3342-50), and ABT331440 (PCT Publication No. WO 02/06223).

Other typical therapeutic agents which may also be included along with an antihistamine include non-steroidal anti-inflammatory drugs (NSAIDs), steroids and antibiotics (e.g., antibacterial and antifungal). NSAIDs include aspirin, acetaminophen, phenylpropionic derivatives (e.g., ibuprofen, naproxen), oxicams (e.g., piroxicam), ketorolac, celecoxib and rofecoxib. Steroids included for use in the present invention include momethasone, dexamethasone, butoxicart, rofleponide, budesonide, deflazacort, ciclesonide, fluticasone, beclomethasone, betamethasone, Fluocinolone, prednisone, prednisolone, loteprednol or triamcinolone. Antibacterial agents include β-lactam antibiotics (e.g., penicillin, amoxicillin, cloxacillin, dicloxacillin, methicillin, nafcillin, oxacillin and piperacillin), aminoglycosides (e.g., amikacin, gentamicin, kanamycin, neomycin, netilmicin, streptomycin and tobramycin), macrolides, lincomycin, and clindamycin, tetracyclines (e.g., demeclocycline, doxycycline, minocycline, oxytetracycline, tetracycline), quinolones (e.g., cinoxacin, nalidixic acid), fluoroquinolones (e.g., iprofloxacin, enoxacin, grepafloxacin, levofloxacin, lomefloxacin, norfloxacin, ofloxacin, sparfloxacin, trovafloxacin), polypeptides (e.g., bacitracin, colistin, polymyxin B), solfonamides, trimethoprim-sulfamethoxazole (TMP-SMX), chloramphenicol, vancomycin, quinupristin/dalfopristin, metronidazole, rifampin, spectinomycin and nitrofurantoin. Antifungals for use in the present invention include posaconazole, voriconazole, ketoconazole, fluconazole, itraconazole, saperconazole, neticonazole, oxiconazole, isoconazole, sulconazole, terconazole, ravuconazole, capsofungin, tioconazole, and/or the pharmaceutically acceptable salts thereof.

Any of these additional ingredients, including salts thereof and other therapeutic agents from the same therapeutic classes, are suitable for inclusion in the syrups of the present invention.

Suitable non-sugar based artificial sweetening agents for use in the present invention include sucralose, a fluorinated sucrose derivative, saccharin, nutritive dextrose, acesulfame potassium, saccharin, aspartame, and mono-ammonium glycyrrhizinate (Magnasweet™). Particularly preferred are sucralose and monoammonium glycyrrhizinate. The sweetening agent may be present in amounts such as, for instance, about 0.01% to about 10%, preferably about 0.1% to about 1%.

Magnasweet™ (commercially available from International Flavors & Fragrances), is the mono-ammonium salt of a triterpenoid saponin derived from the licorice root.

Typically, suitable pharmaceutically acceptable solvents and/or carrier systems include water, alcohols and glycols, especially propylene glycol, sorbitol, ethanol, polyethylene glycol and/or glycerin. The liquid pharmaceutical compositions indicated for pediatric use should be substantially free of and most preferably should not contain ethanol. Use of a combination of at least one of water, propylene glycol, sorbitol and glycerin is preferred. Propylene glycol may be present in a concentration of about 50 to 200 mg/mL. Sorbitol may be present in a concentration of about 100 to 250 mg/mL. Normally, the pharmaceutically acceptable liquid carrier is purified water.

Suitable buffer systems of use in the present invention include, by way of example only, citric, tartaric, fumaric, maleic, phosphoric, and acetic acids and salts. Preferred buffering systems include citric acid and phosphoric acid buffer systems. The citric acid buffer system preferably contains sodium citrate in combination with citric acid. Preferably there is about 0.1 to about 10 grams/liter of sodium citrate, and about 0.05 to about 5 grams/liter of citric acid. Typically suitable buffer systems include those capable of maintaining a pH in the range of greater than about 4.5, preferably about 4.5 to about 6.5, more preferably about 5 to about 6, more preferably about 5.5.

Suitable thickening agents for use in the present invention include, inter alia, guar gum, gelatin, locust bean gum, tara gum, xanthan gum, tamarind gum, tragacanth gum, karaya gum, konjac mannan, a water-soluble carboxyvinyl polymer (e.g., povidone), sodium carboxymethylcellulose, sodium alginate, pectin, azotobacter vinelandii gum, carrageenan, polyethylene glycol, modified starch, cassia gum, psyllium seed gum, carboxymethylcellulose, hydroxypropyl cellulose, hydroxypropyl methylcellulose, hydroxyethyl cellulose, methyl cellulose and microcrystalline cellulose.

In sugar based syrup formulations it is often desirable to employ antimicrobial preservatives. The amount of a pharmaceutically acceptable preservative required to protect a syrup against microbial growth varies with the proportion of water available for growth, the nature and inherent preservative activity of some formulative materials (as many flavoring oils and co-solvents such as propylene glycol are inherently sterile and possess antimicrobial activity), and the capability of the preservative itself. Among the preservatives commonly used in the preservation of syrups with the usually effective concentrations are benzoic acid (0.1 to 0.5%), sodium benzoate (0.1 to 0.5%), and various combinations of methyl-, propyl-, and butylparabens (totaling about 0.1%). In another aspect of the present invention, it has been found that sodium benzoate is not necessary for certain embodiments of to the present invention.

Stabilizers may also be incorporated into the syrup formulation. Useful aminopolycarboxylic acids and salts thereof are those which are safe for ingestion and have sufficient solubility in the syrup formulations to make a stable single phase composition. Commercially available compounds which could be used include iminodiacetic acid, methyliminodiacetic acid, nitrilotriacetic acid, ethylenediaminetetraacetic acid (“EDTA”), diethylenetriaminepentaacetic acid, 1,2-diaminocyclohexane-tetraacetic acid, N-hydroxyethylenediaminetriacetic acid and related compounds. Mixtures of two or more of the foregoing are suitable for use. From the aspects of ready availability, safety, efficacy and cost, the alkali metal salts of EDTA are presently preferred. In those embodiments containing a stabilizer, the stabilizer may be present in amounts of about 0.01 to about 5%, preferably about 0.25%. In an alternative embodiment of the present invention, EDTA is not a necessary ingredient.

Preferably, the formulations of the present invention have less than 0.2% desloratadine degradation products over time under accelerated stability testing, more preferably less than 0.1%. Preferably, the formulations of the present invention are stable at 6 months under accelerated stability testing conditions, more preferably greater than a year, more preferably greater than 15 months and most preferably greater than two years. In addition to being stable, the syrups should not discolor as is known to one of skill in the art.

Most syrups are flavored with synthetic flavorants or with naturally occurring materials such as volatile oils (e.g., orange oil), vanillin, and others, to render the syrup pleasant tasting. Because syrups are aqueous preparations, these flavorants must possess sufficient water-solubility. Typical flavoring agents which are commonly used in sweetened pharmaceuticals, foods, candies, beverages are also useful in the present invention; these materials may impart flavors such as flavor red fruits, green apple, grape cherry, citrus, peach, strawberry, bubble gum, peppermint and many others are within the scope of the present invention. Preferred flavoring agents are Flavor Red Fruits 700-14-01 and Green Apple Flavor.

Also provided by the invention are methods for treating and/or preventing allergic and inflammatory conditions of the skin or airway passages in humans in need of such treating and/or preventing which comprise administering an effective amount of a desloratadine or loratadine. The phrase “allergic and inflammatory conditions of the skin or airway passages” as used herein means those allergic and inflammatory conditions and symptoms found on the skin and in the upper and lower airway passages from the nose to the lungs. Typical allergic and inflammatory conditions of the skin or upper and lower airway passages include seasonal and perennial allergic rhinitis, allergic rhinitis associated with cough, non-allergic rhinitis, asthma including allergic and non-allergic asthma, sinusitis, colds, bronchopulmonary conditions of allergic origin associated with cough, where viscosity and mucous adherence are increased, obstructing permeability of the airways, acute, chronic, spasmodic and asthmatic bronchitis, bronchial asthma, bronchiectasis, sinusitis, otitis media, pneumonia; bronchopneumonia, atelectasis by mucous obstruction, and dermatitis, especially allergic and atopic dermatitis, and urticaria and symptomatic dermographism as well as retinopathy, and small vessel diseases, associated with diabetes mellitus.

Prior art syrup formulations of desloratadine oral solution such as that disclosed in U.S. Pat. No. 6,514,520 have been manufactured as follows: Desloratadine and flavor (Natural & artificial flavor for bubblegum, #15864) are dissolved in propylene glycol. The remaining formulation excipients are dissolved in water. The propylene glycol concentrate is added to the aqueous vehicle with mixing. Water is added qs ad final volume. When the resulting formulation is stored under dark conditions, a strong pink color has been observed to develop over time. This color formation may derive from interaction between desloratadine and the flavorant or between the desloratadine and propylene glycol or between desloratadine and stainless steel. It was necessary to add a yellow dye to mask the color change in the prior art syrup formulation. There exists an additional need for novel processes for producing clear syrups that are sugar free and dye free. As illustrated in the following Examples, the present invention provides syrups that are sugar free and dye free and that do not substantially discolor over time.

Accordingly, the invention will be further described by means of the following examples, which are not intended to limit the scope of the invention as defined by the appended claims.

### EXAMPLE 1

| Ingredient | Concentration (mg/mL) |
|---|---|
| Desloratadine, Micronized | 0.5 |
| Propylene Glycol | 100 |
| Sorbitol Liquid | 150 |
| Sodium Citrate Dihydrate | 2 |
| Citric Acid Anhydrous | 0.64 |
| Povidone K 29/32 | 5 |
| Sucralose | 0.5 |
| Flavor Red Fruits 700-114-01 | 1 |
| Sodium Benzoate | 1 |
| Edetate Disodium | 0.25 |
| Water, purified | q.s. |
| | 1 ml |

### EXAMPLE 2

| Ingredient | Concentration (mg/mL) |
|---|---|
| Desloratadine, micronized | 0.25 |
| Propylene glycol | 100 |
| Sorbitol liquid | 150 |
| Sodium Citrate Dihydrate | 1.26 |
| Citric Acid Anhydrous | 0.5 |
| Mono-ammonium glycyrrhizinate | 7.5 |
| (e.g., Magnasweet ™ Powder) | |
| Green Apple Flavor | 0.5 |
| Sodium Benzoate | 1 |
| Edetate Disodium | 0.25 |
| Water, purified | q.s. |
| | 1 ml |

To prepare the above syrup formulations, the ingredients with the exception of desloratadine are dissolved or mixed into a vessel as is known to one of skill in the art. The addition to the manufacturing process of the dissolving of the desloratadine directly into the finished formulation that incorporates all of the remaining ingredients listed in the above formula avoids the contact between desloratadine and the propylene glycol and bubble gum flavor solution that may have produced a pink color in the prior art formulations that needed to be color masked with a yellow dye.

### Stability Protocol

Three batches of the exemplary formulation detailed in Example 1 were manufactured (referred to herein as Batches A, B, and C) and packaged in two different sizes of amber glass bottles (120 mL and 15 mL). Samples from each of the three batches packaged: in two different sizes was evaluated for pH stability, physical appearance, desloratadine stability, total degradation product stability, sodium benzoate stability, EDTA assay stability, photostability and freezer thaw stability under the following long-term, intermediate and accelerated stability storage conditions and testing frequency,

Storage Conditions and Testing Frequency

| Storage Condition | Time Point |
|---|---|
| Photostability test | 1.2 million Lux hours |
| Freezer thaw (−20° C./25° C.) | 24, 48 and 72 h |
| Refrigeration (0 to 5° C.) | 3, 6, 9 and 12 months |
| 25° C./60% RH | 3, 6, 9, 12, 18, 24 and 36 months |
| 30° C./65% RH | 3, 6, 9, 12, 18, 24 and 36 months |
| 40° C./75% RH | 1, 2, 3, 6 months |

### pH Stability

The pH data from all samples show a good stability trend throughout the 18 months stability interval (see Table 1). The pH values ranged from 5.55 to 5.63 in samples stored at refrigeration (0 to 5° C.), 5.54 to 5.66 in samples stored at 25° C./60% RH and 5.57 to 5.68 in samples stored at 30° C./65% RH.

### Table 1

TABLE 1
Results of pH

| | | pH | | | | | |
|---|---|---|---|---|---|---|---|
| | | Specification | | | | | |
| | | 5 to 6 | | | | | |
| | | Batches | | | | | |
| | | A | | B | | C | |
| | | Volume | | | | | |
| | | 120 | 15 | 120 | 15 | 120 | 15 |
| Stability Conditions | | mL | mL | mL | mL | mL | mL |
| Time point | Temperature(° C.)/ | Initial | | | | | |
| (months) | RH | 5.57 | 5.58 | 5.60 | 5.61 | 5.58 | 5.60 |
| 1 | 40° C. ± 2° C./ | 5.59 | 5.63 | 5.59 | 5.62 | 5.61 | 5.63 |
| | 75% ± 5% RH | | | | | | |
| 2 | 40° C. ± 2° C./ | 5.54 | 5.59 | 5.58 | 5.60 | 5.57 | 5.61 |
| | 75% ± 5% RH | | | | | | |
| 3 | Ref (0-5° ± 2° C.) | 5.60 | 5.61 | 5.60 | 5.61 | 5.60 | 5.63 |
| | 25° C. ± 2° C./ | 5.58 | 5.63 | 5.61 | 5.61 | 5.61 | 5.66 |
| | 60% ± 5% RH | | | | | | |
| | 30° C. ± 2° C./ | 5.60 | 5.64 | 5.60 | 5.64 | 5.60 | 5.62 |
| | 65% ± 5% RH | | | | | | |
| | 40° C. ± 2° C./ | 5.60 | 5.65 | 5.60 | 5.64 | 5.61 | 5.65 |
| | 75% ± 5% RH | | | | | | |
| 6 | Ref (0-5° ± 2° C.) | 5.55 | 5.59 | 5.57 | 5.59 | 5.58 | 5.59 |
| | 25° C. ± 2° C./ | 5.58 | 5.62 | 5.58 | 5.62 | 5.59 | 5.63 |
| | 60% ± 5% RH | | | | | | |
| | 30° C. ± 2° C./ | 5.62 | 5.63 | 5.62 | 5.63 | 5.60 | 5.64 |
| | 65% ± 5% RH | | | | | | |
| | 40° C. ± 2° C./ | 5.62 | 5.66 | 5.61 | 5.66 | 5.62 | 5.67 |
| | 75% ± 5% RH | | | | | | |
| 9 | Ref (0-5° ± 2° C.) | 5.57 | 5.59 | 5.58 | 5.59 | 5.58 | 5.59 |
| | 25° C. ± 2° C./ | 5.54 | 5.61 | 5.57 | 5.61 | 5.58 | 5.60 |
| | 60% ± 5% RH | | | | | | |
| | 30° C. ± 2° C./ | 5.58 | 5.61 | 5.59 | 5.61 | 5.57 | 5.62 |
| | 65% ± 5% RH | | | | | | |
| 12 | Ref (0-5° ± 2° C.) | 5.57 | 5.59 | 5.58 | 5.59 | 5.58 | 5.60 |
| | 25° C. ± 2° C./ | 5.56 | 5.62 | 5.57 | 5.62 | 5.58 | 5.62 |
| | 60% ± 5% RH | | | | | | |
| | 30° C. ± 2° C./ | 5.59 | 5.62 | 5.59 | 5.63 | 5.60 | 5.63 |
| | 65% ± 5% RH | | | | | | |
| 18 | 25° C. ± 2° C./ | 5.59 | 5.62 | 5.63 | 5.66 | 5.61 | 5.62 |
| | 65% ± 5% RH | | | | | | |
| | 30° C. ± 2° C./ | 5.66 | 5.65 | 5.61 | 5.63 | 5.64 | 5.68 |
| | 65% ± 5% R | | | | | | |

### Physical Appearance

Color data showed a change in chromaticity coordinates (a, b). At 25° C./60% RH and 30° C./65% RH, the value of “a*” changed from positive to negative data (see FIGS. 1A and 1B) and the value of “b*” increased to 2.53 (see FIGS. 1C and 1D). The changes in chromaticity coordinates indicated a slight change from a green to red-yellow solution which is not perceived by the naked eye. The intensity (L*) remained constant (see FIGS. 1E and 1F) which indicates that the sample remains a clear solution. Thus, no change in physical appearance was observed (by the naked eye) after storage for up to 18 months at 25° C./60% RH or 30° C./65% RH. The samples were observed to be clear, colorless to yellowish solution, free from foreign matter throughout the 18 months stability interval.

Microbial testing was also conducted on samples at the beginning of the stability study and after 12 months at 30° C./65% RH. The microbial quality was found to be satisfactory. That is, to have a total aerobic microbial count of not more than 100 bacteria/mL, total molds and yeast count of not more than 10 fungi/mL, and absence of E. coli, P. aeruginosa, S aureus, and Salmonella sp.

### Desloratadine Stability

All samples show a good desloratadine stability trend no matter if they are stored at refrigeration (0 to 50° C.), 25° C./60% RH, 30° C./65% RH or 40° C. Data obtained through 18 months at 25° C./60% RH and 30° C./65% RH were evaluated by regression analysis in order to determine a suitable shelf life (see FIGS. 2A and 2B). Based on this regression analysis, the predicted desloratadine assay values over a 24 month shelf life are as follows:
Desloratadine (stored at 25° C./60% RH): 100.9%
Desloratadine (stored at 30° C./65% RH): 98.9%
   Thus, a 24 months shelf life is suitable provided the formulation is stored in amber glass at not more than 30° C.

### Total Degradation Product Stability

Numerical data reported are only those results that exceed the limit of quantitation (LOQ). The LOQ for Desloratadine is 0.03% and the limit of detection (LOD) is 0.007%. In particular, degradation products quantified during stability were Loratadine RS, Loratadine RS LRD-C, DS2 (Desloratadine Imp DS2HCl) and unspecified degradations products. Desloratadine degradation products demonstrated the same behavior no matter if packaged in 15 or 120-ml amber glass bottles.

For samples stored at all stability conditions, Loratadine RS, Loratadine RS LRD-C, DS2 (Desloratadine Imp DS2 HCL) and unspecified degradations products remained within acceptable limits, and the data analysis predicted a maximum level of 0.113% and 0.115% of total degradation products for samples stored at 25° C./60% RH and 30° C./65% RH respectively at the 95% Cl, after storage for 18 months.

DS2 (Desloratadine Imp DS2HCl) is an impurity related substance to raw material and is quantified in all samples at all stability conditions. Samples stored at Photostability and freezer thaw cycle presented a maximum value of 0.108% and 0.102% respectively. Meanwhile samples stored at Refrigeration (0 to 5° C.), 25° C./60% RH, 30° C./65% RH and 40° C./75% RH showed the maximum values of 0.138%, 0.131%, 0.128%, and 0.114% at 6 months respectively.

Data obtained through 18 months at 25° C./60% RH and 30° C./65% RH were evaluated by regression analysis in order to determine product stability over a defined shelf life (see FIGS. 3A and 3B). Based on regression analysis, the to predicted desloratadine total degradation products values over a 24 month shelf life are as follow:
DL Total Deg. Prod, (stored at 25° C./60% RH): 0.113%
DL Total Deg. Prod. (stored at 30° C./65% RH): 0.150%
   Thus, a 24 months shelf life is suitable provided the formulation is stored in amber glass at not more than 30° C.

### Sodium Benzoate Assay and Edetate Disodium (EDTA) Assay

Sodium benzoate and EDTA assay data from samples displayed no degradation trend throughout the 18 month stability interval. Specifically, sodium benzoate data through 18 months at 25° C. ranged from 100.0 to 101.8% and data at 30° C./65% RH ranged from 99.4 to 102.3% of label strength. EDTA data through 18 months at 25° C. ranged from 97.7 to 101.3% and data at 30° C./65% RH ranged form 97.0 to 102.7% of label strength. Additionally, APE Test results obtained at the beginning of the stability study and after 12 months at 30° C./65% RH corroborated that the preservative capacity of sodium benzoate remains satisfactory.

### Photostability Test and Freezer Thaw

Data obtained from samples exposed to the ICH photostability conditions in quartz cells and stressed samples at −20° C./25° C. cycle were found to be within the specification detailed below.

| | |
|---|---|
| Test | Shelf-Life Specification |

Specification

| | |
|---|---|
| pH | 5 to 6 |
| Chromaticity | L*: 98 to 102 |
| | a*: −2.5 to 1 |
| | b*: 0 to 5 |
| Description | Clear, colorless to yellowish solution |
| | free from foreign matter. |

Assay

| | |
|---|---|
| Desloratadine | 0.463 to 0.538 mg/mL |
| | (92.5 to 107.5% LS) |
| Sodium Benzoate | 0.9 to 1.1 mg/mL |
| | (90 to 110% LS) |
| Edetate Disodium | 0.225 to 0.275 mg/mL |
| | (90 to 110% LS) |

Degradation Products

| | |
|---|---|
| Loratadine RS | ≦0.4% |
| Loratadine RS | ≦0.4% |
| LRD-C | |
| DS2 | ≦0.3% |
| Unspecified | ≦0.4% |
| degradation | |
| products | |
| Total degradation | ≦1.5% |
| products | |
| Microbial Limits | Total Aerobic Microbial Count: |
| | Not more than 100 bacteria/mL |
| | Total Molds and Yeast: |
| | Not more than 10 fungi/mL |
| | Absence of: E. coli, P. aeruginosa, |
| | S. aureus, Salmonella sp |
| APE Test | Pass the Test |

Based on the data provided herein the exemplary formulation of the present invention exhibited pH stability, physical appearance stability, desloratadine stability, total degradation product stability, sodium benzoate stability, EDTA assay stability, photostability and freezer thaw stability.

Having described specific preferred embodiments of the invention with reference to the accompanying drawings, it will be appreciated that the present invention is not limited to those precise embodiments and that various changes and modifications can be effected therein by one of ordinary skill in the art without departing from the scope or spirit of the invention as defined by the appended claims.

What is claimed is:

1. An antihistaminic syrup formulation in which the ingredients comprise loratadine, desloratadine, or a pharmaceutically acceptable salt thereof, or a combination of two or more thereof, propylene glycol, sorbitol, sodium citrate dihydrate, citric acid anhydrous, povidone, sucralose, optionally sodium benzoate, and optionally an aminopolycarboxylic acid or salt thereof wherein the antihistaminic syrup formulation has a pH of greater than about 4.5, said antihistaminic syrup formulation being storage-stable.

2. The antihistaminic syrup formulation according to claim 1, wherein the pH is between about 4.5 and about 6.5.

3. The antihistaminic syrup formulation according to claim 1, wherein the pH is between about 5 and about 6.

4. The antihistaminic syrup formulation according to claim 1, wherein the pH is about 5.5.

5. The antihistaminic syrup formulation according to claim 1, wherein propylene glycol is present at about 0.01 to about 35%.

6. The antihistaminic syrup formulation of claim 1, in which the ingredients comprise:

| Ingredient | Concentration (mg/mL) |
|---|---|
| Desloratadine | 0.5 |
| Propylene Glycol | 100 |
| Sorbitol | 150 |
| Sodium Citrate Dihydrate | 2 |
| Citric Acid Anhydrous | 0.64 |
| Povidone | 5 |
| Sucralose | 0.5 |
| Water | q.s. |
| | 1 ml |

7. The antihistaminic syrup formulation of claim 6, in which the ingredients further comprise about 0.1 to about 0.5% sodium benzoate.

8. The antihistaminic syrup formulation of claim 6, in which the ingredients further comprise about 0.01 to about 5% edetate disodium.

9. The antihistaminic syrup formulation of claim 1, which has less than 0.2% desloratadine degradation products after storage for 18 months.

10. The antihistaminic syrup formulation of claim 1, which has less than 0.2% desloratadine degradation products after storage for 24 months.

11. An antihistaminic syrup formulation in which the ingredients comprise loratadine, desloratadine, or a pharmaceutically acceptable salt thereof, or a combination of two or more thereof, propylene glycol, sorbitol, sodium citrate dihydrate, citric acid anhydrous, mono-ammonium glycyrrhizinate, optionally sodium benzoate, and optionally an aminopolycarboxylic acid or salt thereof, wherein the antihistaminic syrup formulation has a pH greater than about 4.5, said antihistaminic syrup formulation being storage-stable.

12. The antihistaminic syrup formulation according to claim 11, wherein the pH is between about 4.5 and about 6.5.

13. The antihistaminic syrup formulation according to claim 11, wherein the pH is between about 5 and about 6.

14. The antihistaminic syrup formulation according to claim 11, wherein the pH is about 5.5.

15. The antihistaminic syrup formulation according to claim 11 wherein propylene glycol is present at about 0.01 to about 35%.

16. The antihistaminic syrup formulation of claim 11, in which the ingredients comprise:

| Ingredient | Concentration (mg/mL) |
|---|---|
| Desloratadine | 0.25 |
| Propylene glycol | 100 |
| Sorbitol | 150 |
| Sodium Citrate Dihydrate | 1.26 |
| Citric Acid Anhydrous | 0.5 |
| Mono-ammonium glycyrrhizinate | 7.5 |
| Water | q.s. |
| | 1 ml |

17. The antihistaminic syrup formulation of claim 16, in which the ingredients further comprise about 0.1 to about 0.5% sodium benzoate.

18. The antihistaminic syrup formulation of claim 16, in which the ingredients further comprise about 0.01 to about 5% edetate disodium.

19. A method for treating and/or preventing allergic and inflammatory conditions of the skin or airway passages in a human in need thereof which comprises administering an effective amount of the antihistaminic syrup formulation of claim 1 or 11.

20. The method of claim 19, wherein an effective amount of the antihistaminic syrup formulation delivers 25 mg or less of propylene glycol for every kilogram of body weight per day.
